(19) United States
(12) Patent Application Publication
(10) Pub. No.: US 2012/0184837 A1
(43) Pub. Date: Jul. 19, 2012

(54) Brain Mapping Probe

(75) Inventors: Hubert Cécile François Martens, Eindhoven (NL); Ke Wang, Valkenswaard (NL)

(73) Assignee: SAPIENS STEERING BRAIN STIMULATION B.V., Eindhoven (NL)

(21) Appl. No.: 13/349,895

(22) Filed: Jan. 13, 2012

(30) Foreign Application Priority Data
Jan. 14, 2011 (EP) 11150938.6

(51) Int. Cl. A61B 5/0478; A61B 5/0484

(52) U.S. Cl. 600/378

(57) ABSTRACT

The invention relates to a probe for stimulation and recording of neural activity in the brain, the probe comprising an axially extending shaft. In one embodiment, at least one stimulation electrode partially encircles the shaft besides at a gap, wherein one or more recording electrodes are located in the gap.

### CROSS-REFERENCE TO RELATED APPLICATIONS

This application claims the benefit of European Patent Application No. 11150938.6 filed on Jan. 14, 2011, which is incorporated herein by reference in its entirety.

### BACKGROUND OF THE INVENTION

The present invention generally relates to a probe for stimulation and recording of neural activity of the brain.

From U.S. Patent Application Publication No. 2007/0123765, a device for brain stimulation is known with a longitudinally extending surface on which a plurality of cylindrical stimulation electrodes is disposed. Axially between two such stimulation electrodes, a plurality of smaller recording electrodes is arranged.

### BRIEF SUMMARY OF THE INVENTION

Based on this background it was an object of the present invention to provide means for the stimulation and recording of neural activity in the brain, wherein it is desirable that physiologically relevant signals can readily be recorded.

This object is achieved by a probe according to claim 1 and a system according to claim 14. Preferred embodiments are disclosed in the dependent claims.

The probe according to the present invention serves for the stimulation and recording of neural activity in the brain, particularly for acute stimulation and recording (i.e. for a short-term use). In some embodiments, the probe comprises the following components:

a) A shaft that extends longitudinally in some “axial” direction. The shaft will typically have a rod-like or filamentary shape and a sufficient stability such that it can be advanced through neural tissue.
b) At least one electrode which will be called “stimulation electrode” in the following because it is intended for delivering electrical stimulation pulses to surrounding neural tissue. The stimulation electrode shall partially encircle the shaft in the form of a ring which is open at (at least one) axially extending gap. Here and in the following, the term “axially” shall refer to the axis of the shaft.
c) At least one electrode which will be called “recording electrode” in the following because it is intended for sensing/recording electrical signals generated in surrounding neural tissue. The at least one recording electrode is disposed in the aforementioned gap of the stimulation electrode (electrically isolated from the latter).

A probe with the above design has the advantage that stimulation as well as recording can be done at substantially the same position without a need to move the probe between a stimulation and a recording event. The arrangement of the stimulation and recording electrodes at the same axial position of the shaft is particularly advantageous in this context (and preferred over an axially displaced arrangement at the same circumferential position of the shaft) because the more elongated axial direction of the probe has a higher probability to be crossing through multiple functional regions. Hence it is important to ensure that recording and stimulation can be performed at substantially equivalent positions, i.e. along the axis of the shaft. Moreover, the area of the stimulation electrode can be enlarged to any practically desired value by increasing its axial extension, wherein such an enlargement has no drawbacks with respect to the spatial matching of stimulation and recording because recording electrodes can readily cover any axial extension of the stimulation electrode.

The gap of the stimulation electrode may extend straightly and parallel to the axial direction. It may however also be locally slanted with respect to the axial direction. The gap may for example “spiral” along the shaft or have a “wavy” shape. Such shapes may have the advantage of generating more homogeneous stimulation fields in axial direction.

Moreover, the gap may have equal or varying width along its extension.

Optionally a plurality of recording electrodes may be disposed in a single gap of a stimulation electrode. Most preferably, a number of two to about ten recording electrodes is provided in the gap of the stimulation electrode.

When a plurality of recording electrodes is disposed in a gap of a stimulation electrode, these recording electrodes are preferably aligned (i.e. disposed one next to the other) in the direction of the gap (e.g. in axial direction for an axially extending gap). In this way it is possible to keep the gap in the stimulation electrode as small as possible.

According to another embodiment of the invention, the probe may comprise a plurality of stimulation electrodes of the kind described, i.e. stimulation electrodes that form an open ring around the shaft with at least one recording electrode being located in their gap. Preferably, a number of two to about eight such stimulation electrodes may be provided on the shaft of the probe. These stimulation electrodes may be distributed uniformly or in any other appropriate pattern along the axis of the probe.

According to a particular embodiment of the aforementioned design, the gaps of the stimulation electrodes are aligned in axial direction.

According to another preferred embodiment of the design with a plurality of stimulation electrodes, the gaps of at least two such stimulation electrodes are rotated with respect to each other (about the axis of the shaft). Most preferably, each gap is rotated with respect to the gaps of all other stimulation electrodes. In this case recording electrodes can be distributed all around the circumference of the probe.

To keep the stimulation site as close as possible to a recording site, the gap in the stimulation electrode will usually be as small as possible. In a preferred embodiment, the gap extends over about 1° to about 45° of the circumference of the shaft, preferably over about 5° to about 30°.

The diameter of the shaft (and hence the corresponding ring-diameter of the stimulation electrode) optionally ranges between about 200 micron and about 2 mm, preferably between about 200 micron and about 1.3 mm, most preferably between about 200 micron and about 600 micron.

The area of a stimulation electrode must be large enough such that this electrode is capable to deliver a desired stimulation current, i.e. that the impedance between the electrode and surrounding neural tissue is not too high. Most preferably, the stimulation electrode has an area between about 0.3 mm² and about 3 mm².

The axial extension of a single stimulation electrode preferably ranges between about 500 and about 2000 micron. With these values, the aforementioned preferred area size of the stimulation electrodes can be achieved for typical shaft diameters.

As the recording electrode shall sense electrical signals of single neuron activity, its size can be (much) smaller than that of a stimulation electrode. Preferably, the recording electrode has an area between about 100 mm² and about 10,000 mm².

The tip of the probe is preferably rounded to minimize or avoid injury during insertion of the probe into neural tissue.

The invention further relates to a brain stimulation and recording system that comprises a probe of the kind described above, i.e. with an axially extended shaft, at least one stimulation electrode that partially encircles the shaft in the form of a ring which is open at a gap, and at least one recording electrode that is disposed in said gap of the stimulation electrode. Moreover, the system comprises a recording module for recording and processing signals from the recording electrodes, said recording module being coupled (by wire or wirelessly) to the recording electrodes, and a stimulation module coupled to the stimulation electrode for delivering electrical pulses (of any kind and shape) to it.

### BRIEF DESCRIPTION OF THE SEVERAL VIEWS OF THE DRAWINGS

These and other aspects of the invention will be apparent from and elucidated with reference to the embodiment(s) described hereinafter. These embodiments will be described by way of example with the help of the accompanying drawings in which:

FIG. 1 schematically shows a probe according to the present invention; and

FIG. 2 shows electrodes of the probe of FIG. 1 in more detail.

Like reference numbers in the Figures refer to identical or similar components.

### DETAILED DESCRIPTION OF THE INVENTION

Brain mapping (recording electrical brain activity and/or applying electrical brain stimulation) is a commonly used technique during neurosurgical procedures, for instance the implantation of DBS (Deep Brain Stimulation) electrodes. Known brain-mapping probes consist of needle-like devices with a single micro-electrode at the tip suited for recording the brain activity and a macro-electrode on the needle shaft for delivery of stimulation. Recording is performed by positioning the micro-electrode tip in the target. For stimulation the tip is retracted in the needle shaft and the larger macro-electrode at the distal end of the needle shaft is advanced towards the target. This is a cumbersome procedure and consumes a lot of time since the needle needs to be advanced multiple times for a complete spatial mapping of the target area and further it is not possible to easily record and stimulate from the same region.

A probe design for acute brain mapping is therefore proposed that comprises a multitude of microelectrode sites for recording purposes distributed along the probe shaft (e.g. on a line), and one or more stimulation electrodes “wrapped” around the probe shaft and having an aperture or gap in the wrapped electrode at the position of the line of micro-electrodes.

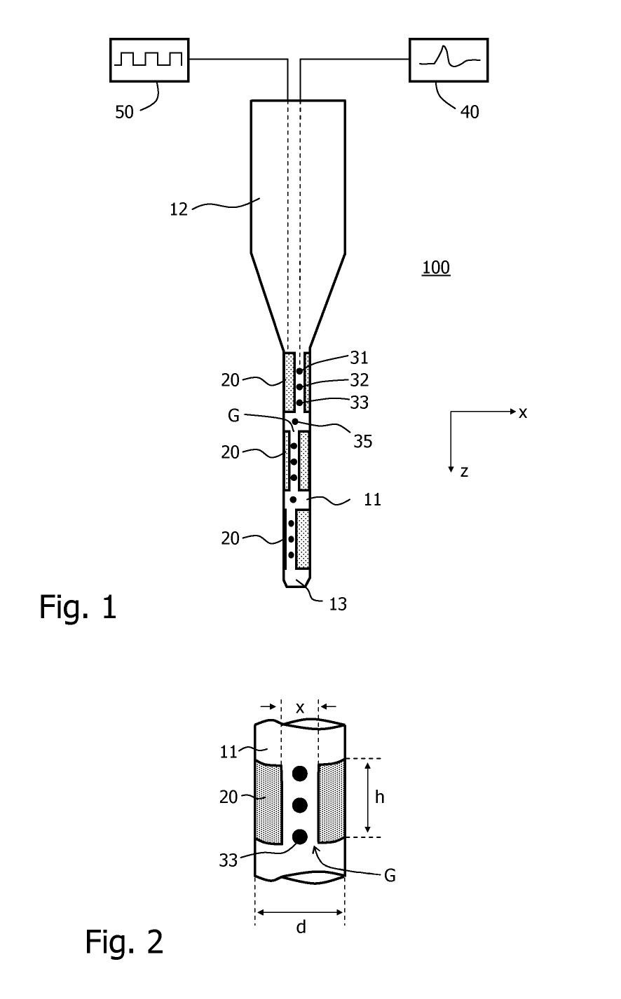

FIGS. 1 and 2 show a probe 100 that is designed according to the aforementioned general principles. The probe 100 comprises a body made of an electrically isolating material (e.g. some polymer), said body consisting of a trunk 12 and a shaft 11 which extends axially in z-direction for insertion into the brain. A typical value for the diameter d of the shaft 11 is about 300 μm.

A plurality of stimulation electrodes 20 is arranged along the axial extension of the shaft 11. Each stimulation electrode 20 has the form of an open cylinder or ring that wraps around the shaft 11, leaving only a gap G of width x open. For the above value of the shaft diameter d, the gap width x typically has a value of about 40 μm. The axial height h of the stimulation electrode 20 may typically have a value of about 1 mm, providing an electrode area of about 0.9 mm². The gaps could optionally also be tilted with respect to the z-axis and/or could have varying width x at different axial positions along their extension.

Within the gaps G of the stimulation electrodes 20, a plurality of recording electrodes 31, 32, 33 is disposed. As can be seen in FIG. 1, the recording electrodes 31-33 in one gap G are aligned in axial direction, while the gaps of different stimulation electrodes 20 are rotated about the axis of the shaft 11 with respect to each other.

Within the shaft 11 and the trunk 12, the stimulation electrodes 20 and the recording electrodes 31-33 are electrically connected to wires (only partially shown) that provide external electrical access to these electrodes. In particular, the stimulation electrodes 20 are connected to a stimulation module 50 that can deliver electrical pulses to them, while the recording electrodes 31, 32, 33, 35 are connected to a recording module 40 that records and processes their signals.

The design of the probe 100 has the advantage that stimulation and recording of neural activity can be done at about the same position, particularly at exactly the same axial position (z-direction) with respect to the shaft 11. If the different circumferential positions of the stimulation electrodes 20 and the recording electrodes 31-33 should matter, the whole probe 100 can readily be rotated about the axis of the shaft 11 in order to match the positions of the recording electrodes with the (previous) position of the associated stimulation electrode and vice versa. Such a rotation can much easier and more precisely be done than an axial displacement of the whole probe 100 which would be needed to sequentially match axially different positions of recording and stimulation electrodes.

The tip 13 of the shaft 11 is preferably rounded in order to minimize or avoid injury during insertion of the shaft into the brain. Moreover, it should be noted that the shaft 11 be provided with further electrodes, for example additional recording electrodes 35 that are not located within a gap of a stimulation electrode. These additional electrodes 35 are preferably located on a common line with the recording electrodes in the gaps G.

In summary, the invention relates to a probe 100 for (particularly acute) stimulation and recording neural activity in the brain, said probe comprising an axially extending shaft 11. At least one stimulation electrode 20 partially encircles the shaft besides at a gap G, wherein one or more recording electrodes 31, 32, 33 are located in said gap G.

The main advantages of the proposed design are:

Recording and stimulation of the same tissue region is possible without probe movement.

Recording and stimulation can be done simultaneously.

The wrapped stimulation electrode allows delivering stimulation current in 3D fashion similar to a regular macro ring-electrode.

The micro-array of recording electrodes allows recording of brain signals similar to normal micro-electrodes.

The interruption of the stimulation electrode by a gap prevents electrical shunting of the recording electrodes (as would be the case for embedded recording electrodes).

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

I/We claim:

1. A probe for stimulation and recording of neural activity of the brain, comprising:
an axially extended shaft;
at least one stimulation electrode that partially encircles the shaft in the form of a ring which is open at a gap; and
at least one recording electrode that is disposed in said gap.

2. The probe according to claim 1, wherein the gap extends parallel to an axial direction and/or that it is locally slanted with respect to the axial direction.

3. The probe according to claim 1 further comprising a plurality of recording electrodes disposed in the gap.

4. The probe according to claim 3, wherein said recording electrodes are aligned along the gap.

5. The probe according to claim 1 further comprises a plurality of stimulation electrodes having recording electrodes in their gaps.

6. The probe according to claim 5, wherein the gaps are aligned in an axial direction.

7. The probe according to claim 5, wherein the gaps of at least two of said stimulation electrodes are rotated with respect to each other.

8. The probe according to claim 1, wherein the gap extends over about 5° to about 45° of the circumference of the shaft.

9. The probe according to claim 1, wherein the shaft has a diameter between about 200 μm and about 1.3 mm.

10. The probe according to claim 1, wherein the stimulation electrode has an area between about 0.3 mm² and about 3 mm².

11. The probe according to claim 1, wherein the stimulation electrode has an axial extension between about 500 μm and about 2000 μm.

12. The probe according to claim 1, wherein the at least one recording electrode has an area between about 100 μm² and about 10000 μm².

13. The probe according to claim 1, wherein the shaft has a rounded tip.

14. A brain stimulation and recording system, comprising:
a probe according to claim 1;
a recording module coupled to the at least one recording electrode for recording signals from the at least one recording electrode; and
a stimulation module coupled to the stimulation electrode for delivering pulses to the stimulation electrode.
